(19) United States
(12) Patent Application Publication
(10) Pub. No.: US 2003/0171737 A1
(43) Pub. Date: Sep. 11, 2003

(54) Soft convex adhesive wafer

(76) Inventors: Walter F. Leise, JR., Lindenhurst, IL (US); Ronald S. Botten, Gurnee, IL (US); Ole Pedersen, Frederiksberg (DK); Christopher T. Glaeser, Grayslake, IL (US)

Correspondence Address: John B. Lungmus, Marshall, Gerstein & Borun, 6300 Sears Tower, 233 South Wacker Drive, Chicago, IL, 60606-6357, US

(21) Appl. No.: 10/095,223

(22) Filed: Mar. 11, 2002

(51) Int. Cl. A61M 1/00; B32B 3/10; B29C 31/00; B29C 33/40

(52) U.S. Cl. 604/540; 428/137; 428/172; 264/40.1; 264/222

(57) ABSTRACT

An adhesive wafer for attaching an ostomy appliance to a peristomal skin surface surrounding a stoma has an integrally molded adhesive layer made of a skin barrier material, a distal surface covered by a flexible backing layer, and a proximal bodyside surface contoured so the adhesive layer has a thin peripheral portion and thick central portion surrounding a stoma-receiving aperture. A set of at least two wafers is provided in one embodiment, wherein individual wafers have differing central portion surface topography, and different stoma receiving apertures. To produce a closely fitting wafer for a given stoma, a method of producing wafers includes providing a representation of the topography of the peristomal surface and the stoma, and, using the representation, manufacturing a mold having a surface configuration substantially matching the topography of the peristomal surface and stoma.

### BACKGROUND

1. Field of the Invention

The present invention relates to a wafer for adhesively attaching an ostomy appliance to a peristomal skin surface surrounding a stoma of a wearer of said appliance.

2. Description of the Prior Art

Within the field of ileostomy, colostomy and urostomy appliances, it is well known that the adhesive wafer should perform a series of important functions such as ensuring secure fastening of the appliance to the skin of the wearer, ensuring a secure sealing function between the exuded stomal fluids and the skin and stoma of the wearer, providing a maximum of comfort and security to said wearer as well as prolonging the wear time of the wafer for economic and practical reasons.

Naturally, because of the inherent severe medical, social, economical and personal problems related to the need for use of an ostomy appliance, any improvement that addresses said problems is an important factor in the quality of life of such wearers and therefore in the consequent health and psychological aspects for said wearer.

Any appreciable improvement of such appliances is therefore of great importance to the increasing number of such wearers.

A large proportion of the problems in connection with ostomy appliances are related to the topography of the peristomal surface surrounding the stoma and of the stoma itself.

On account of a variety of reasons such as initial surgery results, weight increase, muscle relaxation and use of pressure rings in connection with rigid convex wafers or face plates, a groove or moat around the stoma is initially present or develops in a large number of such wearers. These moats can have different shapes, depths and widths and result in difficulties in applying said appliances in a manner that satisfies the requirements as to comfort, adhesion and protection of sensitive skin areas from contact with stomal fluids.

The stoma may protrude more or less, and if the protrusion is small or non-existent, it is necessary to press the adhesive wafer in towards the skin of the wearer so as to cause the stoma to protrude out by pressing the peristomal skin surface inwards. This is usually done with a convex combination of a wafer with substantially uniform thickness deformed to a convex distal bodyside configuration by a stiff convex ring that is attached to a belt so as to exert pressure on the wafer to press the convex bodyside surface thereof caused by the convex ring into the peristomal skin surface of the wearer.

Such a stiff convex ring causes discomfort, is relatively expensive and causes deformation of the peristomal tissue so that formation of a groove or moat around the stoma ensues. This again leads to difficulties in proper sealing around the stoma with ensuing problems and leakage of stomal fluids leading to sores and shorter wear time for the appliance.

To overcome these problems it is known to fill out the moat and seal against the stoma by applying a paste or a separate ring of deformable skin barrier material to the stoma and peristomal skin surface prior to applying the wafer. This is an operation that requires dexterity and is not easy to perform correctly and requires sometimes difficult separate removal of the paste or ring when the wafer is to be replaced.

European patent application No. EP 1 163 892 discloses a deformable ring of skin barrier material that is adhered to the proximal surface of an essentially uniformly thick wafer after being deformed to fit the exterior circumference of the stoma. This solution entails a relatively complicated operation that requires dexterity and therefore is difficult to apply correctly. Because of the various separate elements constituting the combined wafer and ring, it is also relatively expensive.

International patent application No. WO 0053133 discloses a relatively thin adhesive layer of skin barrier material having a thickness of between approximately 0.9 mm and 1.2 mm and shaped to a form a central dome surrounding a stoma receiving aperture and surrounded by a substantially planar peripheral portion. The hollow proximally convex and distally concave dome is deformable because of the relatively thin unreinforced configuration thereof. The deformability of the dome ensures that no appreciable pressure is applied to the peristomal surface and the stoma itself. Furthermore, the ability of the dome surrounding the stoma to absorb moisture without losing the adhesive property of the dome material is relatively limited due to the relatively slight thickness of the adhesive layer.

### SUMMARY OF THE INVENTION

It is a main object of the present invention to provide a wafer of the type indicated that may fit into a moat or groove in the peristomal skin surface surrounding a stoma in a manner providing a certain pressure on said peristomal surface and lateral support pressure on said stoma while at the same time having a relatively large capability of absorbing moisture, being easy to apply and remove and relatively cheap to manufacture.

According to the invention this object is achieved by said wafer comprising an integrally molded adhesive layer consisting of a moisture absorbing and swellable skin barrier material and having a substantially planar distal surface covered by a flexible backing layer for attaching said wafer to a collecting bag and having a proximal bodyside surface that is contoured such that said adhesive layer has a relatively thin peripheral portion and a relatively thick central portion surrounding an aperture for receiving said stoma, the surface area of said central portion being distributed substantially uniformly around said aperture and having a maximum thickness which is at least 2.5 mm larger than the maximum thickness of said peripheral portion.

Advantageously, said peripheral portion has a width of at least 10 mm, preferably at least 12 mm and most preferably at least 15 mm, and the thickness of said peripheral portion is at least 0.4 mm, preferably at least 0,5 mm, more preferably at least 0.6 mm, even more preferably at least 0.7 mm and most preferably at least 0.8 mm.

Hereby, the secure application of the central portion of the bodyside surface of the adhesive layer into a moat surrounding the stoma is ensured in a manner giving a good sealing effect and with exertion of a certain pressure against said peristomal surface and against said stoma.

In the currently preferred embodiment of a wafer according to the invention, said central portion is annular, and said aperture is substantially circular and concentric with said annular central portion.

So as to ensure that good lateral support of the stoma can be obtained, said central portion should have a radial width of at least 6 mm, preferably at least 7 mm and most preferably larger than or equal to 7.5 mm.

So as to ensure that the central portion may fit well around the stoma said annular portion should have a radial width of less than 11 mm, preferably less than 10 mm, most preferably less than or equal to 9.5 mm.

In the currently preferred embodiment of a wafer according to the invention, said central portion has a substantially uniform thickness.

However, so as to accommodate more irregular moat configurations, alternative embodiments are useful where said central portion has a thickness that varies in the circumferential direction and/or in the radial direction.

In another aspect, the present invention relates to a set of at least two wafers for adhesively attaching an ostomy appliance to a peristomal skin surface surrounding a stoma of a wearer of said appliance, each wafer of said set comprising an integrally molded adhesive layer consisting of a moisture absorbing and swellable skin barrier material and having a substantially planar distal surface covered by a flexible backing layer for attaching said wafers to a collecting bag and a proximal bodyside surface that is contoured such that said adhesive layer has a relatively thin peripheral portion and a relatively thick central portion surrounding an aperture for receiving said stoma, said central portion being distributed substantially uniformly around said aperture and having a maximum thickness which is at least 2.5 mm larger than the maximum thickness of said peripheral portion, the individual wafers of said set differing from one another in that the topography of the surface of said central portion is different and optionally in that the size and/or shape of said stoma receiving aperture is different.

In a further aspect, the present invention relates to a method of attaching an ostomy appliance to a peristomal skin surface surrounding a stoma of a wearer of said appliance, the method comprising the following steps:

providing a set of at least two wafers for adhesively attaching said ostomy appliance to said peristomal skin surface, each wafer of said set comprising an adhesive layer consisting of a moisture absorbing and swellable skin barrier material and having a substantially planar distal surface covered by a flexible backing layer for attaching said wafers to a collecting bag and a proximal bodyside surface that is contoured such that said adhesive layer has a relatively thin peripheral portion and a relatively thick central portion surrounding an aperture for receiving said stoma, said central portion being distributed substantially uniformly around said aperture and having a maximum thickness which is at least 2.5 mm larger than the maximum thickness of said peripheral portion, the individual wafers of said set differing from one another in that the topography of the surface of said central portion is different,

evaluating the topography of said persitomal surface and said stoma,

selecting a specific wafer from among the wafers in said set based on said topographical evaluation such that the topography of the surface of said central portion of said specific wafer has the best fit to said topography of said peristomal surface and said stoma, and

locating and adhesively attaching said selected specific wafer on said peristomal surface such that said best fit is exploited.

Hereby, a particularly good fit may be achieved between the peristomal surface and stoma of a specific wearer of an ostomy appliance and the wafer for adhering said appliance.

The added cost of producing, stocking and dispensing a set of differently configured wafers will be more than compensated by the added comfort, wearing time, sealing properties and feeling of security achieved by being able to apply the wafer best suited to the requirements of a specific wearer.

In a yet further aspect, the present invention relates to a method of producing a wafer for attaching an ostomy appliance to a peristomal skin surface surrounding a stoma of a specific wearer of said appliance, said wafer comprising an adhesive layer consisting of a moisture absorbing and swellable skin barrier material and having a distal surface for attaching said wafer to a collecting bag and a proximal bodyside surface for adhering to said peristomal skin surface, the method comprising the following steps:

providing a representation of the topography of said peristomal surface and said stoma,

based on said representation, manufacturing a mold having a surface configuration substantially matching said topography of said peristomal surface and said stoma of said specific wearer, and

utilizing said mold for molding said bodyside surface of said adhesive layer such that a close fit between a wafer molded in said mold and said topography may be achieved.

Hereby, a wafer is provided having as good a fit to the peristomal surface and stoma of a specific wearer as possible. This will maximize the comfort, sealing properties, wearing time and security for the wearer. In view of the impact on wearers of ostomy appliances of having to wear such an appliance as regards the day-to-day quality of life, the added cost of such customized wafers is fully justified. If the total costs of wearing ostomy appliances including hospital treatment for skin and stoma lesions, frequency of replacement, sick leave and so on are taken into account, such added costs for customization may be more than compensated.

A simple embodiment of the method according to the invention comprises the step of producing the representation by making a cast or impression of said peristomal surface and stoma.

The currently preferred method according to the invention comprises the steps of: producing said representation in digital form by scanning said peristomal surface and stoma or by scanning said cast or impression, utilizing said digital representation to produce a mold, and molding said wafer in said mold.

Hereby, the specific wearer of the ostomy appliance is not inconvenienced more than necessary, and no actual physical contact is to be endured as when making a cast or impression. Furthermore, the digital representation may be stored, communicated and amended in many ways.

Preferably, said digital representation is obtained by scanning with x-rays, such as MRI or CATscan, light rays, such as laser, or ultrasound.

In a yet further aspect, the present invention relates to a method of attaching an ostomy appliance to a peristomal skin surface surrounding a stoma of a specific wearer of said appliance by means of a wafer comprising an adhesive layer consisting of a moisture absorbing and swellable skin barrier material and having a distal surface for attaching said wafer to a collecting bag and a proximal bodyside surface for adhering to said peristomal skin surface, the method comprising the following steps:

manufacturing said wafer by means of a method as described in the preceding paragraphs, said representation being a representation of the topography of the peristomal surface of said specific wearer, and

locating and adhesively attaching said wafer on said peristomal surface such that a close fit of said bodyside surface of said wafer to said peristomal surface and stoma is achieved.

In a yet further aspect, the present invention relates to a wafer for adhesively attaching an ostomy appliance to a peristomal skin surface surrounding a stoma of a wearer of said appliance, said wafer comprising an integrally molded adhesive layer consisting of a moisture absorbing and swellable skin barrier material and having a distal surface covered by a flexible backing layer for attaching said wafer to a collecting bag, said flexible backing layer extending at least to the outer periphery of said adhesive layer, the shape of said outer periphery consisting of outwardly convex or substantially rectilinear portions and at least one outwardly concave portion or indentation.

Such an outwardly concave portion or indentation allows a gripping portion of the backing layer to be formed by configuring the backing layer periphery without said indentation such that an area of said backing layer corresponding to the area of said indentation is not adhered to the persitomal skin surface by the adhesive layer and can be gripped for peeling off the adhesive layer from said skin surface when the wafer is to be replaced.

In the currently preferred embodiment of a wafer according to the invention, said outer periphery is generally rectangular and an outwardly concave portion or indentation is located at one of the four corners of said rectangular periphery, preferably at all four corners thereof.

In a yet further aspect, the present invention relates to a mold for producing a wafer for adhesively attaching an ostomy appliance to a peristomal skin surface surrounding a stoma of a wearer of said appliance, said wafer comprising an adhesive layer consisting of a moisture absorbing and swellable skin barrier material and having a sharply defined outer periphery, the mold comprising a first part and a second part adapted for having molding surfaces thereof pressed against one another such that mold hollows in one or both said surfaces together define the configuration of said adhesive layer, at least one periphery defining body for defining said outer periphery being arranged in a groove in a surface of one of the first or second part such that said body is displaceable in the direction towards the other part.

Hereby, a simple means is provided for ensuring a sharply defined periphery of the adhesive layer such that soiling or adhesion of the clothes or bedclothes of a wearer is avoided.

In the currently preferred embodiment of a mold according to the invention, a biasing means is arranged in said groove in one of said first and second parts for biasing said body in said direction towards the other part when said parts are pressed together, and said body is annular and comprises outwardly convex portions and/or substantially rectilinear portions and outwardly concave portions or indentations.

In a final aspect, the present invention relates to a method of producing a wafer for adhesively attaching an ostomy appliance to a peristomal skin surface surrounding a stoma of a wearer of said appliance, said wafer comprising an integrally molded adhesive layer consisting of a moisture absorbing and swellable skin barrier material and having a substantially planar distal surface covered by a flexible backing layer for attaching said wafer to a collecting bag and a proximal contoured bodyside surface having a first surface topography such that said adhesive layer has a relatively thin peripheral portion and a relatively thick central portion surrounding an aperture for receiving said stoma, said proximal surface being covered by a release layer and said central portion having a maximum thickness which is at least 2 mm larger than the maximum thickness of said peripheral portion, preferably at least 3 mm larger than said maximum thickness, the method comprising the steps of:

providing a first mold for pre-forming said release layer and provided with a first mold hollow having a second surface topography similar to, preferably substantially identical to said first surface topography, said hollow being provided with suction apertures communicating with a vacuum source,

providing a second mold for molding said adhesive layer having a first part and a second part adapted for being pressed against one another, said first part being provided with a second mold hollow for defining said first surface topography of said adhesive layer, providing a substantially continuous supply of a sacrificial web,

arranging said skin barrier material on said sacrificial web,

providing a substantially continuous supply of release web,

heating an area of said release web for enhancing the deformability thereof,

locating said heated area in register with said first mold hollow,

applying vacuum to said suction apertures such that said area is sucked into said hollow and thereby deformed such that said area is provided with a third surface topography substantially identical to said second surface topography,

relieving said vacuum application,

displacing said area into register with said second mold hollow such that said third surface topography with said second surface topography,

displacing a portion of said sacrificial web with barrier material thereon into register with said second mold hollow such that said area is located between said portion and said second mold hollow, and

pressing said second part against said first part such that said area and said portion are pressed into said second mold hollow.

Hereby, the release sheet is applied and laminated to the bodyside surface of the adhesive layer in a manner allowing short molding time in the second mold hollow. If the release sheet were formed and laminated to the adhesive layer in the second mold hollow this would require a much longer molding time and consequent lower production rate. The much longer molding time in such case is owing to formation of wrinkles in the release sheet, which requires a long molding pressure duration to remove the wrinkles.

In the following the different aspects of the invention will be described more in detail with reference to different embodiments thereof shown, solely by way of example, in the drawings, where:

### DESCRIPTION OF THE SEVERAL VIEWS OF THE DRAWING

FIGS. 1-5 are diagrammatic perspective views of five different embodiments of a wafer according to the invention,

FIG. 6 is a diagrammatic cross-sectional view taken along line A-A in FIG. 1 and shown in enlarged scale,

FIG. 7 is a schematic illustration of a system for scanning the peristomal skin surface and stoma of a patient and illustration of utilization of the scanning result to provide a mold,

FIG. 8 is a schematic view illustrating the method according to the invention of producing a wafer according to the invention,

FIG. 9 is a diagrammatic, broken away, partially sectional view in enlarged scale of a mold for use in the method illustrated in FIG. 8, and

FIGS. 10 and 11 are diagrammatic bottom plan views of two embodiments of the top mold shown in FIG. 9.

### DETAILED DESCRIPTION OF THE PREFERRED EMBODIMENTS

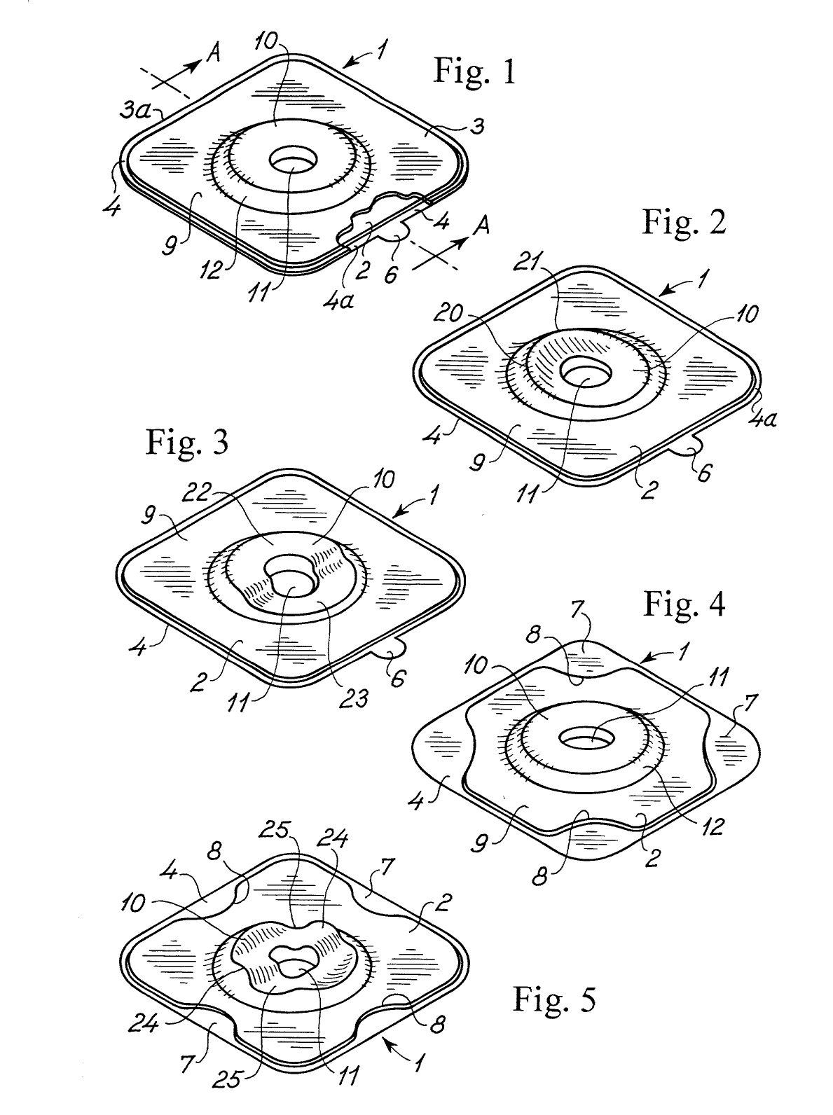

Referring now to FIGS. 1-6, a wafer for adhesively attaching an ostomy appliance to the peristomal skin surface surrounding the stoma of a wearer of the appliance is indicated generally by the reference numeral 1. The wafer 1 comprises an adhesive layer 2 of moldable skin barrier material sandwiched between a release sheet 3 and a flexible backing layer 4 for attaching said wafer 1 to a not shown collecting bag.

The skin barrier material referred to herein may be any of the many skin barrier materials known in the art which is suitable for molding to form the adhesive layer of a wafer according to the present invention.

In FIG. 1 the release sheet 3 has been partially cut away to show the location of the adhesive layer 2 between the release sheet 3 and the backing layer 4. In FIGS. 2-5 the release sheet has been removed for the sake of clarity.

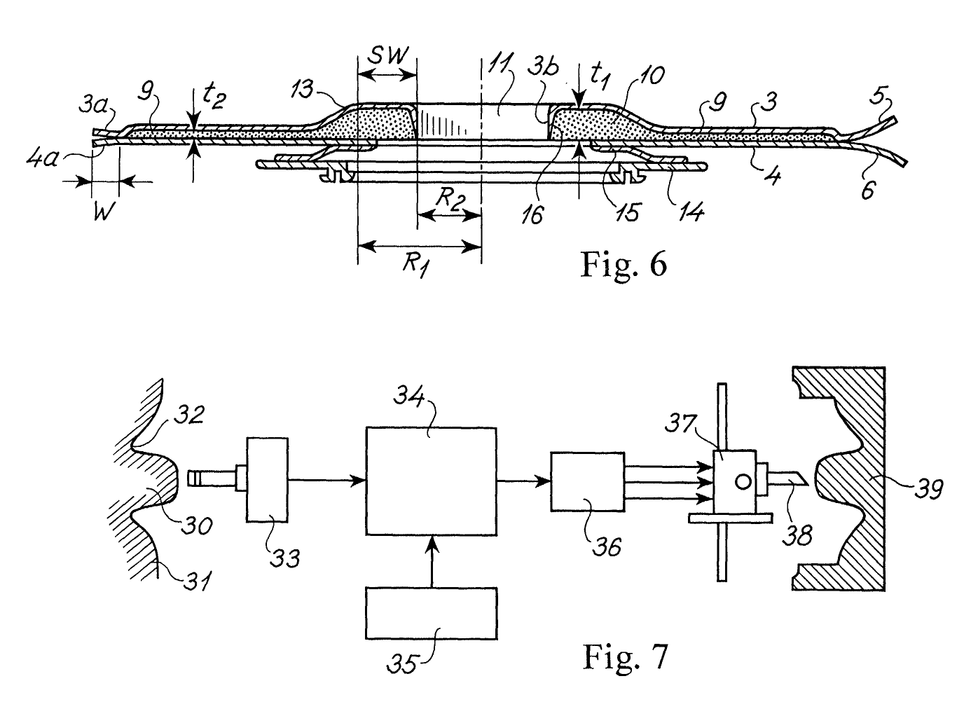

In the embodiments of FIGS. 1-3 both the release sheet 3 and the backing layer 4 are provided with a gripping tab 5 and 6, respectively, see FIG. 6. The gripping tab 5 of the release sheet is intended for use when removing the release sheet 3 from the wafer 1, the release sheet 3 being provided with a coating of silicone or other release agent, such that the release sheet 3 can be removed prior to applying the thus exposed surface of the adhesive layer 2 of the wafer 1 to the peristomal skin surface of said wearer.

The tab 6 of the backing layer 4 is intended for being gripped by the wearer or a health care person when the wafer is to be removed from said adhesion to the peristomal skin surface by peeling the wafer off starting at the region of the adhesive layer 2 adjacent the tab 6.

In the embodiments shown in FIGS. 4 and 5, the tab 6 of the backing layer 4 has been substituted by areas 7 of the backing layer 4 obtained by configuring the periphery of the adhesive layer 2 with indentations or outwardly concave portions 8 such that an area 7 can be gripped for removing the wafer 1. In this manner, relatively large gripping portions 7 are obtained allowing peeling off of the wafer from said peristomal skin surface from various directions which allows greater flexibility in the operation of removing the wafer 1 from said peristomal skin surface.

Portions of the release sheet 3 corresponding to and overlying the portions 7 of the backing layer are intended to serve as gripping portions for removing the release sheet 3 from the adhesive layer 2 prior to applying the wafer 1 to said peristomal skin surface.

The indentations 8 of the periphery of the adhesive layer 8 may have other shapes, particularly in connection with other shapes of the periphery of the release sheet and backing layer, be it triangular, circular or some other shape.

Referring now to FIGS. 1 and 6, the adhesive layer 2 comprises a relatively thin peripheral portion 9 and a relatively thick central portion 10. The central portion 10 is annular and concentric with a substantially circular stoma receiving aperture 11. A tapered portion 12 separates the peripheral portion 9 from the central portion 10.

The central portion 10 has a substantially uniform thickness t1 which is at least 2.5 mm larger than the thickness t2 of the peripheral portion. The substantially planar annular proximal or bodyside surface 13 of the central portion has a width SW, the so-called support width.

An annular coupling ring 14 for coupling the wafer 1 to a not shown collecting bag is attached to the wafer 1 by means of an annular film 15 adhered or sealed to the ring 14 and the backing layer 4. The collecting bag may be attached in other ways to the wafer 1, either removably or non-removably, i.e. so-called two-piece and one-piece ostomy appliances.

Annular edge portions 3a and 4a of the release sheet 3 and backing layer 4, respectively, protrude beyond the periphery of the adhesive layer and have a width W between approximately 2 mm and 5 mm. The edge portion 4a of the backing layer 4 is useful in preventing any flow or leakage of adhesive from the periphery of the adhesive layer 2 from adhering to or soiling the clothes or bed clothes of the wearer.

The release sheet 3 has an inner edge portion 3b covering substantially the entire inner adhesive surface 16 of the stoma receiving aperture 11 such that the adhesive adjacent the surface 16 is protected against drying out and hardening This is important as the surface 16 is intended to sealingly and supportingly surround and contact the not shown protruding stoma of the wearer as explained below.

A number of different wafers constituting a set of wafers are provided for selecting a specific wafer of said set most suited for matching the surface topography of the peristomal skin surface and stoma of a particular wearer of an ostomy appliance comprising said specific wafer. The criteria for selecting said specific wafer are a combination of degree of fit of the annular central portion 10 to a moat or groove in the tissue surrounding the stoma both as regards depth and width thereof, the diameter of the stoma and the necessary degree of lateral support for said stoma.

The wafers in one set of such wafers are therefore differentiated by varying the radius R1 of the annular central portion 10 and the radius R2 of the stoma aperture 11 (and consequently the support width SW) and the thickness t1 of the central portion 10. The thickness t2 of the peripheral portion is preferably constant for all wafers of the set.

### EXAMPLE

In the currently preferred set of wafers of the type shown in FIGS. 1 and 6, the thickness t2 is 0.85 mm. The thickness t2 may be larger or smaller if necessary.

The currently preferred set of wafers of the type shown in FIGS. 1 and 6 are as specified below:

| | | R1 | | | |
|---|---|---|---|---|---|
| No. | Wafer Size (mm × mm) | (mm) | t1 (mm) | R2 (mm) | SW (mm) |
| 1 | 100 × 100 | 22.0 | 3.85 | 12.5 | 9.5 |
| 2 | 100 × 100 | 18.5 | 5.85 | 9.0 | 9.5 |
| 3 | 100 × 100 | 18.5 | 5.85 | 11.5 | 8.0 |
| 4 | 100 × 100 | 22.0 | 5.85 | 12.5 | 9.5 |
| 5 | 100 × 100 | 22.0 | 5.85 | 14.0 | 8.0 |
| 6 | 115 × 115 | 25.0 | 3.85 | 16.0 | 9.0 |
| 7 | 115 × 115 | 25.0 | 3.85 | 17.5 | 7.5 |
| 8 | 115 × 115 | 25.0 | 5.85 | 16.0 | 9.0 |
| 9 | 115 × 115 | 25.0 | 5.85 | 17.5 | 7.5 |
| 10 | 115 × 115 | 27.5 | 5.85 | 19.0 | 8.5 |

The wafer size indicated above is in reality the size of the adhesive layer 2, while the sizes of the wafers 1 including the borders 3a and 4a with a width W of mm are 6 mm larger on each side, i.e. 106 m×106 mm and 121 mm×121 mm, respectively.

The set may also comprise wafers with stoma receiving apertures having a radius R2 of 7.5 mm, these wafers being intended for use by enlarging the stoma receiving aperture by cutting with scissors or the like so as to accommodate wearers with stoma diameters and/or off-center locations that are not entirely accommodated by any of the other wafers of the set.

Furthermore, the set may comprise wafers with elliptical or oval stoma receiving apertures.

It is important to note that the radial width of the bodyside surface 13 or SW should have a minimum size of approximately 6 mm and preferably no smaller than 7 mm and most preferably no smaller than 7.5 mm. If SW is smaller, then the central portion 10 will not support a stoma inserted in the aperture 11 sufficiently in the lateral direction.

SW should not exceed 11 mm, preferably not be larger than 10 mm and most preferably not larger than 9.5 mm because for most configurations of a moat or groove around the stoma of ostomy patients having such a moat, the annular central portion 10 will not fit into the moat and the wafer will not afford correct support for a stoma inserted in the aperture 11 and will not fit well enough to adhere and seal against leakage of stomal fluids into contact with the peristomal skin surface and stoma of the wearer.

Referring now to FIGS. 2-5, further embodiments of the wafer according to the invention are illustrated. The configuration of these embodiments are directed towards accomodating more differentiated surface topographies of the peristomal surface and stoma of a specific wearer.

In the embodiment illustrated in FIG. 2, the thickness of the central portion 10 varies in both the radial and circumferential direction such that a ridge 20 with a peak 21 thereof is formed corresponding to a stoma surrounding moat with a depression having a matching topography.

In the embodiment illustrated in FIG. 3, the thickness of the central annular portion 10 varies circumferentially such that approximately one half 22 of the central portion 10 is substantially thicker than the other half 23, for instance 6 mm and 4 mm, respectively.

In the embodiment illustrated in FIG. 4, the thickness of the central annular portion 10 is uniform as in the embodiment of FIG. 1, but the aperture 11 is oval or elliptical instead of circular so as to accommodate a stoma having an off-center location and/or a non circular cross-section.

In the embodiment illustrated in FIG. 5, the thickness of the central portion varies both in the radial and circumferential direction so that various peaks 24 and throughs 25 are formed in the surface topography of said central portion 10 corresponding to troughs and peaks of the surface topography of the peristomal surface of a specific wearer, said peristomal surface topography being registered and reproduced in inverse relationship as explained in the following with reference to FIG. 7.

The central portion 10 may be configured with an annular periphery as illustrated in FIGS. 1-5, but may also be configured with an irregular periphery corresponding to an irregular periphery of a moat surrounding a stoma of a specific wearer.

All the above described configurations may be included in a set of wafers from which a specific wearer selects the wafer best suited for the special circumstances of said specific wearer. In this manner a specific wearer may identify a standard product included in said set of wafers which will function best. However the wafer may also be customized to fit the specific wearer based on the specific surface topography of the peristomal surface and stoma of said specific wearer as described in the following with reference to FIG. 7.

In FIG. 7, a stoma 30 and a peristomal surface 31 with an exaggerated moat or groove 32 surrounding the stoma are illustrated in cross section.

A scanning mechanism 33 is arranged for scanning the topography of the stoma 30, the surface 31 and the moat 32, the scanning data being transferred to a computer 34 where a digital representation of the topography of the stoma 30 and surrounding skin surface 31 and 32 is created.

A control input device 35 causes the digital representation to be transferred to a digitally controlled activator 36 for causing a shaping device 37 with a cutting tool 38 to shape a die 39 for producing a mold for molding the proximal bodyside surface of a wafer having a substantially perfect fit with the stoma 30 and surrounding skin surface 31, 32.

The process and equipment is illustrated in FIG. 7 in a symbolic manner and represents various possible manners of scanning the peristomal skin surface and stoma, either directly from the wearer or from a cast or impression made by means of wax or other suitable materials as well as various possible manners of providing a mold from said scanning procedure.

The scanning procedure may take place for instance either by means of laser light or ultrasound applied directly to the wearer or by x-rays applied to a cast or impression. Various different procedures to achieve such scanning and conversion into a digital representation and a die or mold are known, inter alia from U.S. Pat. Nos. 4,611,288, 4,663,720, 5,056,204 and 5,487,012, all incorporated herein by reference.

The mold for such customized molding of the proximal surface of the wafer 1 may also be produced in a conventional manner directly from a cast or impression of the peristomal surface and stoma of a specific wearer.

The distal surface of the wafer may be provided with visual or tactile markings or guide lines that indicate the correct orientation of the wafer when being applied to the peristomal surface such that the fit between the proximal surface of the wafer and said peristomal surface is obtained.

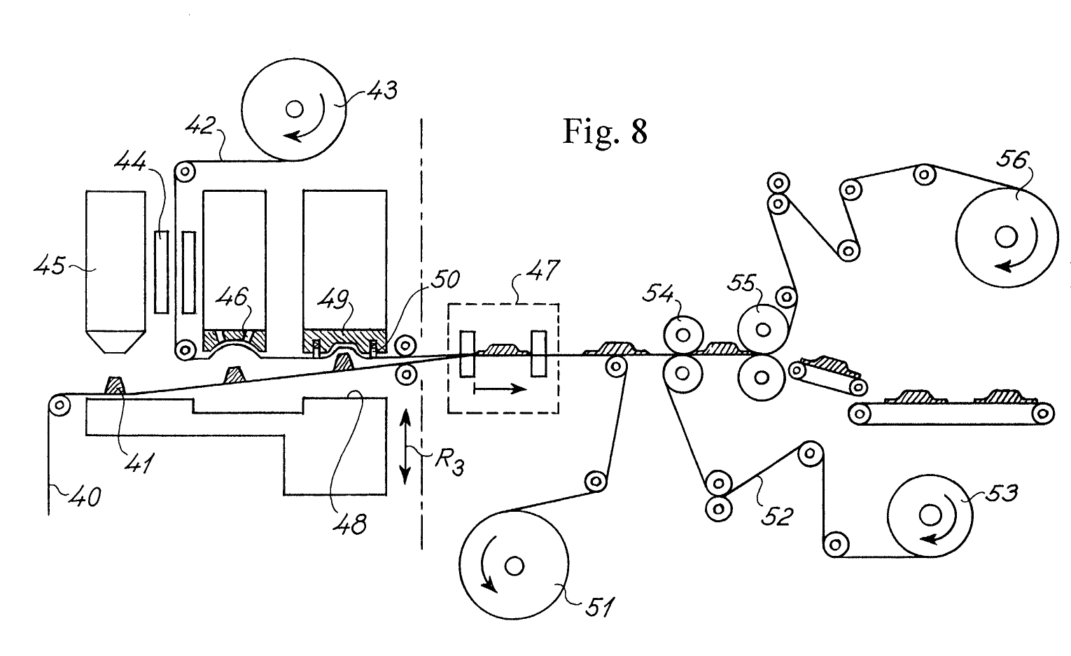

Referring now to FIG. 8, a sacrificial or support web 40 of PET coated with a release agent is supplied from a not shown roll, and patties 41 of a skin barrier material are deposited thereon from a container 45 thereof. A release web 42 with a thickness of approximately 150 m and coated with a release agent such as silicone is supplied from a roll 43 thereof and preheated to approximately 120 degrees centigrade at a heating station 44 and thereafter preformed in a vacuum mold, the web being sucked unto the mold surface by vacuum supplied to channels 46 such that a preformed area of the release liner 42 is obtained.

An indexing station 47 moves the sacrificial web 40 and the release web forward step-wise such that a precise register of webs 40 and 41 takes place relative to one another and the various treatment stations in the process train.

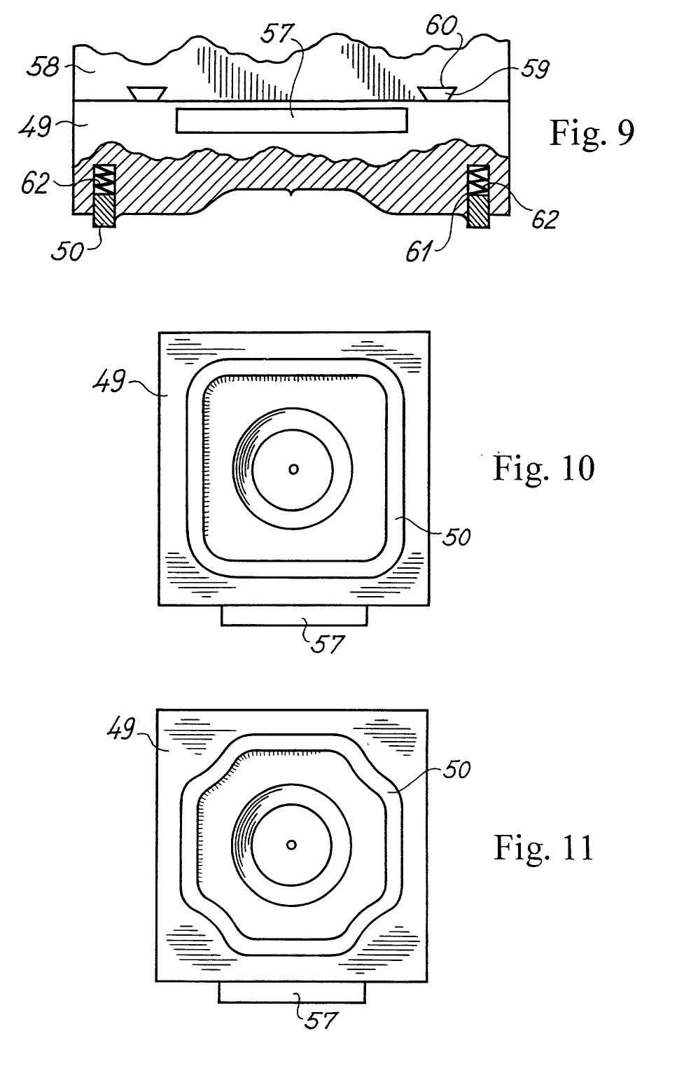

The preformed area of the release web 44 is located centrally over a patty 41 between a lower mold part 48 and an upper mold part 49 provided with a spring loaded annular body 50 described more in detail in the following with reference to FIGS. 9-11. The upper mold 49 has a molding surface very similar to and preferably identical with the molding surface of the vacuum mold 46.

The lower mold 48 is pressed upwards as indicated by the arrow R3 so that the patty 41 and the preformed area of the release web 42 are molded to the desired topography of the proximal surface of the final wafer 1. As the release web 42 has been preformed to the desired surface topography only the patty material is to be molded by the pressure exerted by the movable mold part 48.

After the indexing station 47 the support web 40 is peeled away from the molded patties and wound up on a roll 51, and a corona treated EMA backing web 52 is supplied from a roll 53 thereof and laminated to the distal surface of the adhesive layer (patty) at a laminating station 54. Finally, the finished wafers 1 are cut out at a rolling cutting station cutting through the release web 41 and the backing web 52, the cutting waste of the webs 41 and 52 being wound up on a waste roller 56. The wafers 1 are transported to a not shown punching station where the stoma receiving aperture 11 of the wafer 1 is formed.

Referring now to FIGS. 9-11, the upper mold 49 in FIG. 8 is provided with a handle 57 and is attached to a supporting body 58 by means of two ribs 59 displaceably inserted in corresponding grooves 60 in the supporting body 58 such that the mold 49 can be substituted quickly by another mold. This attachment system is also utilized for the vacuum mold 46 so that this mold also may be replaced quickly.

The annular body 50 is received displaceably in an annular groove 61 and may be displaced upwards in said groove against the force of compression springs 62. Not shown cooperating shoulders on the body 50 and the inner surface of the groove 61 prevent the body 50 from being pressed entirely out of the groove by the springs 62.

When a wafer 1 is being molded in the mold 48, 49, the body 50 functions as a stop 1 ring to prevent barrier material of the patty 41 from being pressed beyond the stop ring 50 that is kept pressed against the release web 42 and the lower mold 48 by the springs 62 during the molding operation whereby a sharply defined periphery of the adhesive layer of the wafer is formed and no adhesive is pressed onto the ring 4a of backing material (see FIG. 6).

In FIG. 10, stop ring 50 corresponding to the periphery of the adhesive layer 2 in FIGS. 1-3 is shown, while the stop ring 50 shown in FIG. 11 corresponds to the periphery shown in FIG. 4.

While several embodiments of the different aspects of the invention have been described, other embodiments are conceivable without departing from the scope of the invention as defined by the appended patent claims.

We claim:

1. A wafer for adhesively attaching an ostomy appliance to a peristomal skin surface surrounding a stoma of a wearer of said appliance, said wafer comprising an integrally molded adhesive layer consisting of a moisture absorbing and swellable skin barrier material and having a substantially planar distal surface covered by a flexible backing layer for attaching said wafer to a collecting bag, and a proximal bodyside surface that is contoured such that said adhesive layer has a relatively thin peripheral portion and a relatively thick central portion surrounding an aperture for receiving said stoma, a surface area of said central portion being distributed substantially uniformly around said aperture and having a maximum thickness which is at least 2.5 mm larger than the maximum thickness of said peripheral portion.

2. A wafer according to claim 1, wherein said peripheral portion has a width of at least 10 mm, preferably at least 12 mm and most preferably at least 15 mm.

3. A wafer according to claim 1 or 2, wherein the thickness of said peripheral portion is at least 0.4 mm, preferably at least 0,5 mm, more preferably at least 0.6 mm, even more preferably at least 0.7 mm and most preferably at least 0.8 mm.

4. A wafer according to any if the preceding claims, wherein said central portion is annular.

5. A wafer according to claim 4, wherein said aperture is substantially circular and concentric with said annular central portion.

6. A wafer according to any of the claims 1-5, wherein said central portion has a radial width of at least 6 mm, preferably at least 7 mm and most preferably larger than or equal to 7.5 mm.

7. A wafer according to any of the claims 1-6, wherein said central portion has a radial width of less than 11 mm, preferably less than 10 mm, most preferably less than or equal to 9.5 mm.

8. A wafer according to any if the preceding claims, wherein said central portion has a substantially uniform thickness.

9. A wafer according to any of the claims 1-7, wherein said central portion has a thickness that varies in the circumferential direction.

10. A wafer according to any of the claims 1-7 or 9, wherein said central portion has a thickness that varies in the radial direction.

11. A set of at least two wafers for adhesively attaching an ostomy appliance to a peristomal skin surface surrounding a stoma of a wearer of said appliance, each wafer of said set comprising an adhesive layer consisting of a moisture absorbing and swellable skin barrier material and having a substantially planar distal surface covered by a flexible backing layer for attaching said wafers to a collecting bag and a proximal bodyside surface that is contoured such that said adhesive layer has a relatively thin peripheral portion and a relatively thick central portion surrounding an aperture for receiving said stoma, a surface area of said central portion being distributed substantially uniformly around said aperture and having a maximum thickness which is at least 2.5 mm larger than the maximum thickness of said peripheral portion, the individual wafers of said set differing from one another in that the topography of the surface of said central portion is different.

12. A set of wafers according to claim 11, wherein at least two of the wafers of the set is in accordance with any of the claims 2-10.

13. A set of wafers according to claim 11 or 12, wherein at least two wafers differ from one another in that the size and/or shape of said stoma receiving aperture is different.

14. A method of attaching an ostomy appliance to a peristomal skin surface surrounding a stoma of a wearer of said appliance, the method comprising the following steps:
providing a set of at least two wafers for adhesively attaching said ostomy appliance to said peristomal skin surface, each wafer of said set comprising an adhesive layer consisting of a moisture absorbing and swellable skin barrier material and having a substantially planar distal surface covered by a flexible backing layer for attaching said wafers to a collecting bag and a proximal bodyside surface that is contoured such that said adhesive layer has a relatively thin peripheral portion and a relatively thick central portion surrounding an aperture for receiving said stoma, said central portion being distributed substantially uniformly around said aperture and having a maximum thickness which is at least 2.5 mm larger than the maximum thickness of said peripheral portion, the individual wafers of said set differing from one another in that the topography of the surface of said central portion is different, evaluating the topography of said peristomal surface and said stoma,
selecting a specific wafer from among the wafers in said set based on said topographical evaluation such that the topography of the surface of said central portion of said specific wafer has the best fit to said topography of said peristomal surface and said stoma, and
locating and adhesively attaching said selected specific wafer on said peristomal surface such that said best fit is exploited.

15. A method according to claim 14, wherein said set of wafers is in accordance with any of the claims 11-13.

16. A method of producing a wafer for attaching an ostomy appliance to a peristomal skin surface surrounding a stoma of a specific wearer of said appliance, said wafer comprising an adhesive layer consisting of a moisture absorbing and swellable skin barrier material and having a distal surface for attaching said wafer to a collecting bag and a proximal bodyside surface for adhering to said peristomal skin surface, the method comprising the following steps:
providing a representation of the topography of said peristomal surface and said stoma,
based on said representation, manufacturing a mold having a surface configuration substantially matching said topography of said peristomal surface and said stoma of said specific wearer, and
utilizing said mold for molding said bodyside surface of said adhesive layer such that a close fit between a wafer molded in said mold and said topography may be achieved.

17. A method according to claim 16, wherein said method comprises the step of producing the representation by making a cast or impression of said peristomal surface and stoma.

18. A method according to claim 16 or 17, wherein said method comprises the steps of:
producing said representation in digital form by scanning said peristomal surface and stoma or by scanning said cast or impression,
utilizing said digital representation to produce a mold, and
molding said wafer in said mold.

19. A method according to claim 18, wherein said digital representation is obtained by scanning with x-rays, light rays or ultrasound.

20. A method of attaching an ostomy appliance to a peristomal skin surface surrounding a stoma of a specific wearer of said appliance by means of a wafer comprising an adhesive layer consisting of a moisture absorbing and swellable skin barrier material and having a distal surface for attaching said wafer to a collecting bag and a proximal bodyside surface for adhering to said peristomal skin surface, the method comprising the following steps:
manufacturing said wafer by means of a method according to any of the claims 16-19, said representation being a representation of the topography of the peristomal surface of said specific wearer, and
locating and adhesively attaching said wafer on said peristomal surface such that a close fit of said bodyside surface of said wafer to said peristomal surface and stoma is achieved.

21. A wafer for adhesively attaching an ostomy appliance to a peristomal skin surface surrounding a stoma of a wearer of said appliance, said wafer comprising an integrally molded adhesive layer consisting of a moisture absorbing and swellable skin barrier material and having a distal surface covered by a flexible backing layer for attaching said wafer to a collecting bag, said flexible backing layer extending at least to the outer periphery of said adhesive layer, the shape of said outer periphery consisting of outwardly convex or substantially rectilinear portions and at at least one outwardly concave portion or indentation

22. A wafer according to claim 21, wherein said outer periphery is generally rectangular and an outwardly concave portion or indentation is located at one of the four corners of said rectangular periphery, preferably at all four corners thereof.

23. A mold for producing a wafer for adhesively attaching an ostomy appliance to a peristomal skin surface surrounding a stoma of a wearer of said appliance, said wafer comprising an adhesive layer consisting of a moisture absorbing and swellable skin barrier material and having a sharply defined outer periphery, the mold comprising a first part and a second part adapted for having molding surfaces thereof pressed against one another such that mold hollows in one or both said surfaces together define the configuration of said adhesive layer, at least one periphery defining body for defining said outer periphery being arranged in a groove in a surface of one of the first or second part such that said body is displaceable in the direction towards the other part.

24. A mold according to claim 23, wherein a biasing means is arranged in said groove in one of said first and second parts for biasing said body in said direction towards the other part when said parts are pressed together.

25. A mold according to claim 23 or 24, wherein said body is annular and comprises outwardly convex portions and/or substantially rectilinear portions and/or outwardly concave portions or indentations.

26. A method of producing a wafer for adhesively attaching an ostomy appliance to a peristomal skin surface surrounding a stoma of a wearer of said appliance, said wafer comprising an integrally molded adhesive layer consisting of a moisture absorbing and swellable skin barrier material and having a substantially planar distal surface covered by a flexible backing layer for attaching said wafer to a collecting bag and a proximal contoured bodyside surface having a first surface topography such that said adhesive layer has a relatively thin peripheral portion and a relatively thick central portion surrounding an aperture for receiving said stoma, said proximal surface being covered by a release layer and said central portion having a maximum thickness which is at least 2 mm larger than the maximum thickness of said peripheral portion, preferably at least 3 mm larger than said maximum thickness, the method comprising the steps of:
providing a first mold for pre-forming said release layer and provided with a first mold hollow having a second surface topography similar to, preferably substantially identical to said first surface topography, said hollow being provided with suction apertures communicating with a vacuum source,
providing a second mold for molding said adhesive layer having a first part and a second part adapted for being pressed against one another, said first part being provided with a second mold hollow for defining said first surface topography of said adhesive layer,
providing a substantially continuous supply of a sacrificial web,
arranging said skin barrier material on said sacrificial web,
providing a substantially continuous supply of release web,
heating an area of said release web for enhancing the deformability thereof,
locating said heated area in register with said first mold hollow,
applying vacuum to said suction apertures such that said area is sucked into said hollow and thereby deformed such that said area is provided with a third surface topography substantially identical to said second surface topography,
relieving said vacuum application,
displacing said area into register with said second mold hollow such that said third surface topography with said second surface topography,
displacing a portion of said sacrificial web with barrier material thereon into register with said second mold hollow such that said area is located between said portion and said second mold hollow, and
pressing said second part against said first part such that said area and said portion are pressed into said second mold hollow.
